(19) United States
(12) Patent Application Publication
(10) Pub. No.: US 2003/0125790 A1
(43) Pub. Date: Jul. 3, 2003

(54) Deployment device, system and method for medical implantation

(76) Inventors: Vitaly Fastovsky, Haifa (IL); Orit Yarden, Givat Shmuel (IL); Yehiel Burstein, Kibbutz Hanita (IL); Yoseph Rozenman, Tel Aviv (IL)

Correspondence Address: G.E. EHRLICH (1995) LTD., c/o ANTHONY CASTORINA, SUITE 207, 2001 JEFFERSON DAVIS HIGHWAY, ARLINGTON, VA, 22202, US

(21) Appl. No.: 10/228,268

(22) Filed: Aug. 27, 2002

Related U.S. Application Data
(60) Provisional application No. 60/342,389, filed on Dec. 27, 2001.

(51) Int. Cl. A61F 2/06; A61F 11/00

(52) U.S. Cl. 623/1.11; 606/108

(57) ABSTRACT

A deployment device for deploying a self-expansible medical implant at a target location in a body cavity, is provided. While generally, the expansion of self-expansible structures tends to be abrupt, and the impact of expansion may cause injury, a two-stage expansion process of the present invention minimizes the impact of exapnsion. Additionally, an ability to manuever the medical imlant into position, after the first stage of expansion, provides for accurate positioning.

Thus the present invention is of a deployment device for precise and well-controlled manner of deployment, so as to minimize damage to the cavity wall and to position the implant accurately at the target location.

The deployment device includes: an inner tube; an outer tube; and an implant received on the inner tube and enclosed by the outer tube. The implant has a self-expansible anchoring element which is in a contracted condition when enclosed by the outer tube, expands to a partially-expanded condition when the outer tube is retracted, and expands to a fully-expanded condition when the inner tube is removed.. The implant is deployed by introducing the deployment device to the target location in the body cavity; retracting the outer tube with respect to the implant such that the anchoring element self-expands from its contracted condition to its partially-expanded condition; and withdrawing the inner tube from the implant such that the anchoring element self-expands from its partially-expanded condition to its fully-expanded condition to firmly fix the implant at the target location within the body cavity.

### FIELD AND BACKGROUND OF THE INVENTION

The present invention relates to a deployment device, system and method for deploying a medical implant at a target location in a body cavity.

For various purposes, it is often desired or necessary to implant permanently or for a limited time period, a medical device (referred to herein as implant) in a body cavity. Examples of various types of medical devices which are presently implanted include stents, deflectors, filters, sensors, septal occluders, coils for aneurysm and the like. The implant is in a contracted condition when delivered to the target location in the cavity, e.g., a blood vessel or other lumen, and is expanded at the target location in order to fix it in place.

Often implants are delivered to their intrabody position via an endoluminal procedure, by the use of a catheter based delivery and deployment device navigated into the target cavity aided by a guide-wire which is prepositioned in the body lumen. The catheter is designed to accommodate the wire, typically within a dedicated central lumen.

One common type of implants are force-expansible implants, which are delivered via a delivery and deployment device which includes a balloon or a mechanical mechanism which is inflated or expanded in order to forcibly expand the implant and fix it in the body lumen.

Another common type of implants are self-expansible implants, namely implants which are in a contracted condition as they are delivered to the target location, and then self-expand, once released, to fix it in the target location. Self-expansible implants or anchoring portions thereof are typically made of a shape memory alloy which undergoes a transformation from a contracted, strained shape, to an expanded, memorized shape when heated to body temperature. Such implants are typically propagated to their target location within a constraining tube or sheath of a delivery and deployment device, which constrains the implant in a contracted condition during its delivery to the target location, and which is then removed to permit the implant to self-expand and thereby become fixed when deployed at the target location.

The precise positioning and orienting of the medical implant in the target location of the body cavity are both critical. In addition, the implant should be expanded in a manner which minimizes the possibility of damage to the cavity wall, particularly where the cavity is a blood vessel. It is also desirable to permit some maneuvering of the implant at the target location for precise positioning and orientation of the implant. These tasks become difficult when self-expansible implants inherently having a spring like action when let expand are to be precisely deployed at a target location. The present invention provides solutions to this technological problem.

### OBJECTS AND BRIEF SUMMARY OF THE INVENTION

Hence, an object of the present invention is to provide a deployment device and system, and also a deployment method, having advantages in the above respects for deploying a medical implant at a target location in a body cavity, such as a blood vessel or other body lumen, bladder and the like.

According to one aspect of the present invention, there is provided a deployment system for deploying a medical implant at a target location in a body cavity, comprising: an inner tube; an outer tube being translatably accepted in the outer tube; and an implant received on the inner tube and enclosed by the outer tube; the implant having a self-expansible anchoring element which is in a contracted condition when enclosed by the outer tube, expands to a partially-expanded condition when the outer tube is retracted, and expands to a fully-expanded condition when the inner tube is removed. The inner tube is designed to translatably accept a guide wire.

According to further features in the preferred embodiments of the invention described below, the deployment system further includes a retainer, to prevent the implant from sliding along the inner tube, together with the retracting outer tube.

In the described preferred embodiments, the retainer is an intermediate tube between the inner tube and the outer tube. As an alternative, however, the retainer could be an annular shoulder formed on the inner tube.

According to further features in the described preferred embodiments, a portion of the anchoring element is wrapped, coiled around, hooked, inserted into slots, or otherwise fixed to the inner tube, both in the partially-expanded condition and in the contracted condition of the anchoring element. In one described embodiment, the anchoring element is an elastic spring wire; in another described embodiment, it is an elastic spring leaf.

According to further features in one described embodiment, the deployment system further includes a guide wire to be passed through the inner tube for guiding the implant, inner tube, and outer tube, to the target location in the body cavity. Where the lumen is a blood vessel, the inflated balloon may be used also to propel the deployment device by blood flow to the target location within the blood vessel.

According to further features in one described embodiment, the implant comprises a power source.

According to further features in one described embodiment, the implant comprises an extracorporeally energizeable power source.

According to further features in one described embodiment, the implant is capable of telemetric communication with an extracorporeal device.

According to another described embodiment, the implant is a stent.

According to another embodiment, the deployment system could include a balloon which in inflatable corresponding to the size of the cavity at the target location to thereby properly locate the device at that location by a wedging action.

It will be appreciated that the system components herein described may be broken to a deployment device which includes the tubes, balloon, etc.; the implant and the guide wire. It is contemplated that the deployment device forms another aspect of the invention.

According to another aspect of the present invention, there is provided a method of deploying an implant at a target location in a body cavity, introducing a deployment device as described above to the target location in the body cavity; retracting the outer tube with respect to the implant such that the anchoring element of the implant self-expands from its contracted condition to its partially-expanded condition; and withdrawing the inner tube from the implant such that the anchoring element of the implant self-expands from its partially-expanded condition to its fully-expanded condition and becomes deployed at the target location within the body cavity.

After retracting the outer tube to self-expand the anchoring element from its contracted condition to its partially-expanded condition, the inner tube may be manipulated, if desired, to adjust the position or orientation of the implant within the body cavity before withdrawing the inner tube to fully-expand the anchoring element.

When the term “anchoring element” is used in the singular, it is to be understood that it is intended also to cover the plural. Thus, in most cases there would be a plurality of such anchoring elements.

Generally, the expansion of self-expansible medical implants tends to be abrupt, and the impact of expansion may cause injury to a cavity wall. The present invention successfully addresses the shortcomings of presently known configurations by providing a deployment device for deploying a self-expansible medical implant, by a two-stage expansion process, so as to minimize the impact of exapnsion. Additionally, an ability to manuever the medical implant into position, after the first stage of expansion, provides for accurate positioning.

Thus the present invention is of a deployment device for precise and well-controlled manner of deployment, so as to minimize damage to the cavity wall and to position the implant accurately at the target location.

Further features and advantages of the invention will be apparent from the description below.

### BRIEF DESCRIPTION OF THE DRAWINGS

The invention is herein described, by way of example only, with reference to the accompanying drawings, wherein:

FIG. 1 diagrammatically illustrates one form of medical implant to be deployed in accordance with the present invention, the implant being illustrated in the fully-expanded condition of its anchoring elements;

FIGS. 2a-2c diagrammatically illustrate three stages in preparing a deployment device in accordance with the present invention for deploying the implant at a target location in a body cavity;

FIGS. 3a-3f illustrate another deployment device constructed in accordance with the present invention and six stages in the use of such a device for deploying a medical implant;

FIG. 4 is a flow chart illustrating a method of deploying a medical implant at a desired location in a body cavity in accordance with the present invention;

FIGS. 5a and 5b illustrate the fully-expanded condition and partially-expanded condition, respectively, of a medical implant having another type of anchoring element for implanting in accordance with the present invention; and

FIG. 6 illustrates another construction of fixing the medical implant onto the inner tube.

### DESCRIPTION OF PREFERRED EMBODIMENTS

The present invention is of a deployment device for deploying a self-expansible medical implant, at a target location in a body cavity. While generally, the expansion of self-expansible structures tends to be abrupt, and the impact of expansion may cause injury, a two-stage expansion process of the present invention minimizes the impact of exapnsion. Additionally, an ability to manuever the medical imlant into position, after the first stage of expansion, provides for accurate positioning.

Thus the present invention is of a deployment device for precise and well-controlled manner of deployment, so as to minimize damage to the cavity wall and to position the implant accurately at the target location.

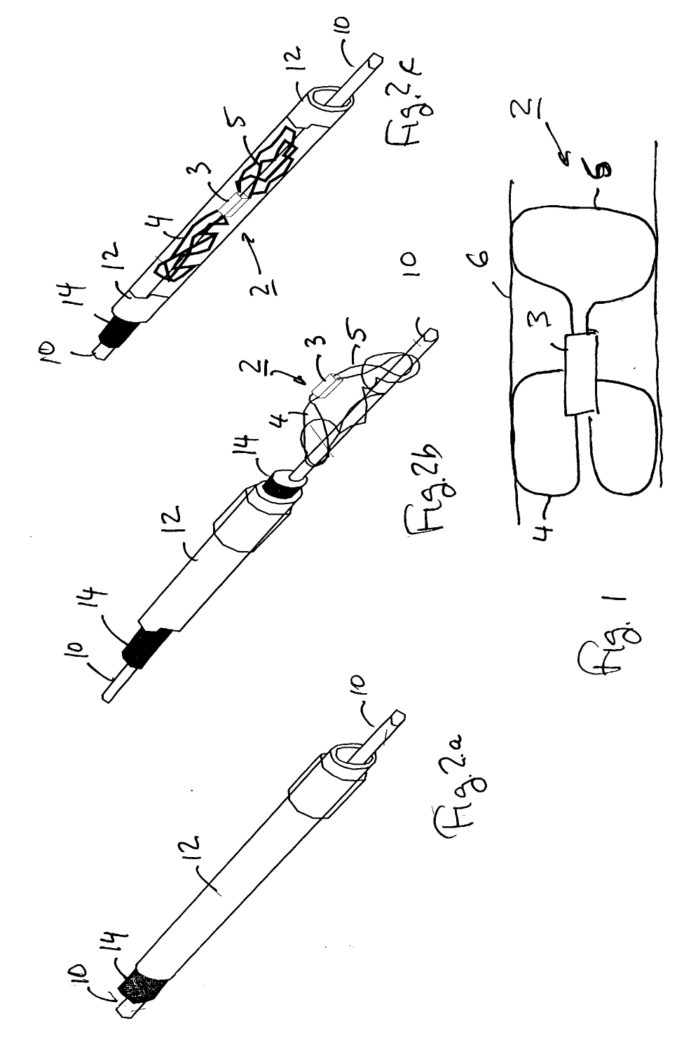

The implant may be, for example, a stent, a filter, a sensor (e.g., pressure, flow-rate, temperature, oxygen concentration) a septal occluder, a coil, a detachable coil for aneurysm treatment, a graft, a deflector, or any other device for performing or measuring a physiological function or parameter within a body cavity. The body cavity in which the implant is to be deployed may be a blood vessel, other body lumen, or other body cavity. FIG. 1 diagrammatically illustrates one medical implant to be deployed in accordance with the present invention, the implant being shown in its fully deployed condition. The illustrated implant, generally designated 2, includes a sensor 3 having a pair of self-expansible anchoring elements 4, 5, which, when fully expanded as shown in FIG. 1, firmly anchor the sensor 3 within the body lumen 6 or other body cavity. In the implant, illustrated in FIG. 1, the anchoring elements 4, 5 are elastic spring wires or struts in the shape of open loops and secured to the opposite ends of the sensor 3. FIG. 1 illustrates the anchoring elements 4, 5 in their fully expanded condition for fixing the sensor within the body cavity or lumen 6, but as described more particularly below, these anchoring elements can also be compacted into a partially-contracted condition as shown in FIG. 2b, or in a fully-contracted condition as shown in FIG. 2c.

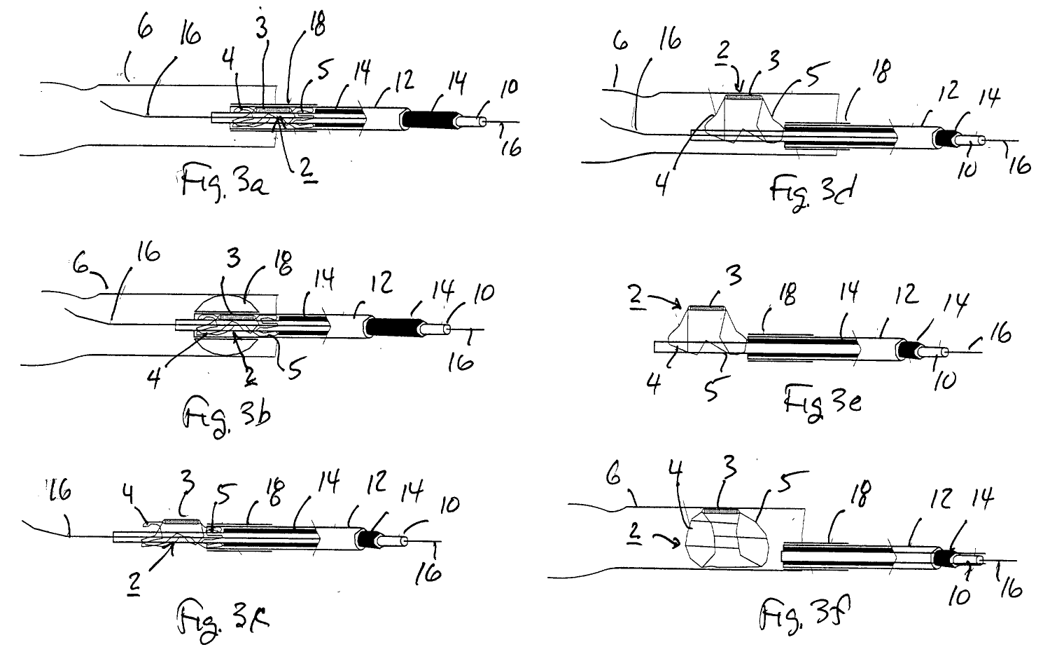

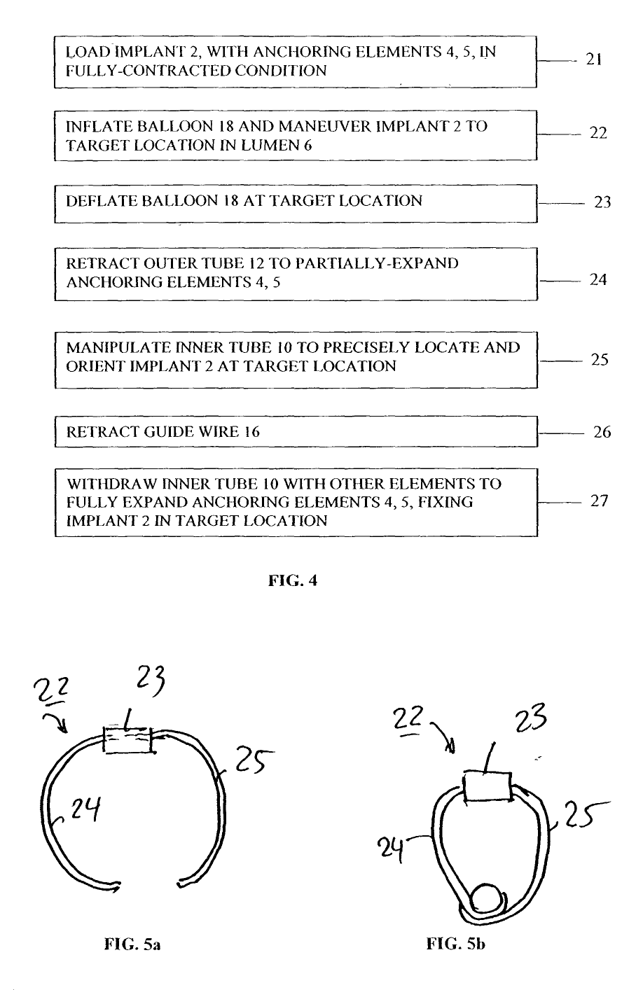

The deployment device for deploying the medical implant 2 of FIG. 2 is more particularly illustrated in FIGS. 2a-2c, and also in FIGS. 3a-3f.

Thus, as shown particularly in FIG. 2b, the deployment device includes an inner tube 10 and an outer tube 12 enclosing the inner tube 10. The medical implant 2 is applied to the inner tube 10 with the self-expansible anchoring elements 4, 5 of the implant in a partially-expanded condition; and then the outer tube 12 is slipped over the implant 2, and the inner tube 10, to constrain the anchoring elements 4, 5 in a fully contracted condition as shown in FIG. 2c.

As will be described more particularly below, especially with respect to FIGS. 3a-3f, the deployment device as illustrated in FIG. 2c, with the outer tube 12 confining the medical implant 2 to its fully contracted condition, is introduced into the body cavity and manipulated to the target location within it, at which time the outer tube 12 is retracted to permit the anchoring elements 4, 5 to assume their partially-expanded condition. The inner tube 10 is then retracted to permit the anchoring elements to assume their fully-expanded condition, firmly fixing the sensor 3 within the body cavity 6, as shown in FIG. 1.

As the outer tube 12 is retracted, it exerts frictional forces on the implant 2. In order to prevent the implant 2 from sliding along the inner tube 10, together with the retracting outer tube 12, the deployment device further includes an annular retainer 14, shown particularly in FIG. 2b, at the distal end of the deployment device receiving the implant 2 in its partially-expanded condition. Retainer 14 in FIGS. 2a-2c is in the form of an intermediate tube between the inner tube 10 and the outer tube 12. The retainer 14 ensures that the implant 2 remains in its designated position along the inner tube 10. It will be appreciated, however, that the retainer 14 could also be in the form of an annular shoulder or flange fixed to the outer surface of the inner tube 10.

When the outer tube 12 is retracted, the retainer flange 14 retains the partially-expanded implant 2 on the inner tube 10 until the inner tube 10 is retracted, whereupon the anchoring elements 4, 5 assume their fully-expanded condition firmly fixing the sensor 3 within the body cavity 6.

As indicated above, the anchoring elements 4, 5 in the medical implant 2 illustrated in FIG. 1 are in the form of elastic spring wires or struts. Such anchoring elements may be mounted in their partially-expanded condition on the inner tube 10 by wrapping, coiling, or fixing portions of the elastic spring wires to or around the outer surface of the inner tube 10, as shown particularly in FIG. 2b.

A deployment device as described above for deploying a medical implant 2 may be guided to its target location in the body cavity by a guide wire. Such a deployment device may also include an inflatable balloon at its distal end for propelling the deployment device by blood flow (e.g., when the body cavity is a pulmonary artery). Such a balloon, when inflated to a size corresponding to the size of the lumen or body cavity at the target location, may also be used to locate the medical implant at the target location, and (or) for wedging the flow in order to measure wedge pressure.

FIG. 3 illustrates a deployment device constructed in accordance with the present invention, and also including a balloon at its distal end, for deploying a medical implant to a target location within a body cavity. To facilitate understanding, those elements of the medical implant which have been described above in FIG. 1, and of the deployment device described above with respect to FIGS. 2a-2c, are identified by the same reference numerals. FIG. 3a illustrates the main elements of such a deployment device, whereas FIGS. 3b-3f illustrate the manner in which such a deployment device is used for deploying the medical implant 2 at the target location within the body cavity.

Thus, as shown in FIG. 3a, the medical implant 2 includes a sensor 3, or other device to be implanted, and a pair of anchoring elements 4, 5 in the form of elastic wire loops partially wrapped around the inner tube 10 (as shown in FIG. 2b), and enclosed by the outer tube 12 to constrain the anchoring elements 4, 5 in their fully contracted condition, (as shown in FIG. 2c). The deployment device shown in FIG. 3a further includes the retainer 14 at the distal end of the inner tube 10, the guide wire 16 which is passed through the inner tube 10 for guiding the deployment device through the lumen to the target location, and the balloon 18 fixed to the distal end of the outer tube 12 for either propelling the deployment device to the target location within the lumen, and/or for locating the deployment device at the target location by inflating the balloon to the diameter of the body cavity at the target location, and (or) for wedging the flow in order to measure wedge pressure.

FIG. 3a illustrates the condition of the deployment device, therein generally designated 20, as it is introduced into the body cavity via the guide wire 16, and with the anchoring elements 4, 5 of the medical implant 2 in their fully contracted conditions as constrained by the outer tube 12.

FIG. 3b illustrates the condition of the deployment device after the balloon 18 has been inflated for propelling the deployment device through the body cavity by the blood flow, and/or for properly locating, by a wedging action, the deployment device at the target location within the body cavity according to the diameter of the inflated balloon, and (or) for measuring the wedge pressure.

After the deployment device has been properly located at its target location, the balloon 18 is deflated and the outer tube 12 is partially retracted. This allows the anchoring elements 4, 5 to assume their partially-expanded condition, as shown in FIG. 3 and more particularly in FIG. 2b.

With the anchoring elements 4, 5 in their partially-expanded condition, the inner tube 10 may now be manipulated to more precisely locate or orient the medical implant 2 at the precise target location within the body cavity 6, as shown in FIG. 3d. When the medical implant has been precisely located and oriented, the guide wire 1 is withdrawn, as shown in FIG. 3e. The inner tube 10, the outer tube 12, retainer 14 and balloon 18, are now withdrawn from the body cavity 6, permitting the anchoring elements 4, 5 to assume their fully-expanded condition firmly fixing the sensor 3 within the body cavity 6, as shown in FIG. 3f.

The foregoing stages in the deployment of the medical implant 2, as illustrated in FIGS. 3a-3f, are indicated by the flow chart illustrated in FIG. 4. Thus, block 21 in FIG. 4 illustrates the stage shown in FIG. 3a wherein the deployment device 20 is loaded with the medical implant 2 in the fully-contracted condition of its anchoring elements 4, 5, and with the balloon 18 deflated; block 22 illustrates the stage shown in FIG. 3b wherein the balloon 18 is inflated; and used for locating the deployment device at the target location; block 23 illustrates the stage shown in FIG. 3f, wherein the balloon 18, after locating the deployment device 20 at the target location, is deflated, and the outer tube 12 is partially retracted to permit the anchoring elements 4, 5 to assume their partially-expanded condition; block 24 illustrates the stage shown in FIG. 3d wherein the inner tube 10 is manipulated to permit precisely locating and orienting the medical implant at the target location; block 25 illustrates the stage shown in FIG. 3e wherein the guide wire 16 is retracted; and block 26 illustrates the stage shown in FIG. 3f, wherein the inner tube 10, the outer tube 12, the retainer 14, the guide wire 16, and the balloon 18, are all withdrawn from the lumen, permitting the anchoring elements 4, 5 of the medical implant 2 to assume their fully expanded condition, firmly fixing the sensor 3 in the body cavity 6.

It will be appreciated that the medical implant 2 illustrated in FIG. 1 is merely one example of a medical implant that can be deployed using the deployment device and method described above. FIG. 5 illustrates another construction of medical implant having self-expansible anchoring elements that may be deployed in this manner.

Thus, the medical implant illustrated in FIG. 5a, and therein generally designated 22, includes a sensor 23 having self-expansible elements 24, 25 in the form of elastic spring leafs of a C-configuration and mounting the sensor 23 at a mid-portion thereof. When such a medical implant is used, the spring leafs 24, 25 may also be wrapped or coiled around the inner tube 10 of the deployment device when received within the outer tube 12 (not shown), such that the outer tube constrains the anchoring elements 24, 25 in their fully contracted condition in the same manner as described above with respect to the elastic spring wires 4, 5 shown in FIG. 1.

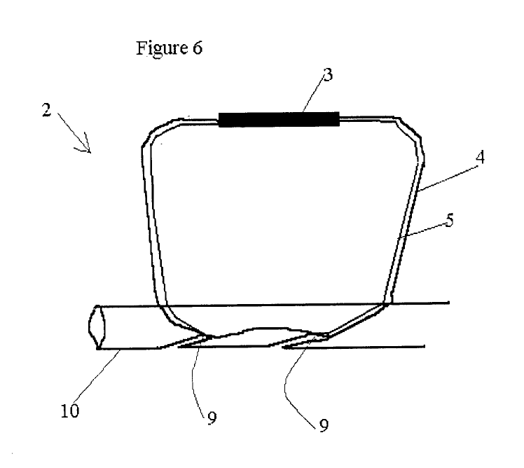

It will be appreciated that the manner of fixing and retaining the anchoring elements 4, 5 of the medical implant 2, illustrated in FIGS. 2a-2c, is merely an example. According to the present invention, the anchoring elements 4, 5 may be wrapped, coiled around, hooked, inserted into slots, or otherwise fixed to the inner tube 10 both in their partially-expanded and in their contracted conditions. FIG. 6 illustrates another construction of fixing the medical implant 2 onto the inner tube 10, by inserting the anchoring elements 4, 5 into slots. Accordingly, the inner tube 10 includes at least one, and preferably two or more slots 9, wherein the anchoring elements 4, 5 may be inserted. The slots 9 are operative to fixate the wrapping of the anchoring elements 4, 5 to a desired site on the inner tube 10. Additionally, the slots 9 prevent the implant 2 from sliding along the inner tube 10, together with the retracting outer tube 12, so that the annular retainer 14 (FIGS. 2a-2c) need not be used.

In accordance with the present invention, medical implant 2, being or having a self-expansible anchoring element, may be a stent.

The deployment device may include many other features. For example, the inner and outer tubes 10 and 12 may have side holes at any location for flushing, and/or for injection of fluids. Such tubes may also have additional lumens for various other purposes, such as for balloon inflation, fluid injection, etc.

In accordance with the present invention, the body cavity may be a blood vessel, a chamber of a heart, a ventricle of a heart, an airway passage, a uterus, a bowl, a digestive tract, or any other body cavity or lumen.

Furthermore, in accordance with the present invention, the body cavity may be a human body cavity or an animal body cavity.

Additionally, in accordance with the present invention, medical implant 2 comprises at least one implant, for example, of the following: a pressure sensor, a flow rate sensor, a temperature sensor, an oxygen concentration sensor, an ion concentration sensor, an impedance sensor, a sensor adapted for cardiac output assessment, a filter, such as a blood filter, a septal occluder, a coil, a detachable coil for aneurysm treatment, a graft, a deflector, and any other device for performing or measuring a physiological function or parameter within a body cavity.

Furthermore, in accordance with the present invention, medical implant 2 may comprise a plurality of sensors and (or) medical implants.

In accordance with the present invention, medical implant 2 may be an energizeable device. Specifically, medical implant 2 may be acoustically energizeable, and include an acoustic transducer, such as a piezoelectric transducer. Alternatively, or additionally, medical implant 2 may be electromagnetically energizeable and include a ferroelectric element. Alternatively or additionally, medical implant 2 may be magnetically energizeable and include a magnet. Alternatively, or additionally, medical implant 2 may be radio frequency energizeable and include a radio frequency antenna or coil and a capacitor.

For example, medical implant 2 may be that described in commonly owned U.S. patent application Ser. No. 09/872129 (Publication No. 20010026111), to Doron et al., “Acoustic biosensor for monitoring physiological conditions in a body implantation site,” incorporated herein by reference. U.S. patent application 20010026111 describes an acoustic biosensor for deployment at an implantation site within a body, such as an abdominal aortic aneurysm. The biosensor includes a sensor element for measuring a physiological condition at the implantation site, and for generating an information signal representative of the physiological condition. The biosensor further includes a piezoelectric transducer element for converting an externally originated acoustic interrogation signal into energy for operating the sensor, and for modulating the interrogation signal, e.g., by employing a switching element to alternate the mechanical impedance of the transducer element, to transmit the information signal outside of the body.

Additionally or alternatively, medical implant 2 may include a piezoelectric transducer, described in commonly owned U.S. Pat. No. 6,140,740 to Porat, et al “Piezoelectric transducer,” incorporated herein by reference. U.S. Pat. No. 6,140,740 describes a miniature piezoelectric transducer element, comprising; (a) a cell element having a cavity; (b) a flexible piezoelectric layer attached to the cell member, the piezoelectric layer having an external surface and an internal surface, the piezoelectric layer featuring such dimensions so as to enable fluctuations thereof at its resonance frequency upon impinging of an external acoustic wave; and (c) a first electrode attached to the external surface and a second electrode attached to the internal surface of the piezoelectric layer. At least one of the electrodes may be specifically shaped so as to provide a maximal electrical output, wherein the electrical output may be current, voltage or power. A preferred shape of the electrodes includes two cores interconnected by a connecting member. The transducer element may function as a transmitter. When used as a transmitter, the electrodes are electrically connected to an electrical circuit including a switching element for modulating the reflected acoustic wave by controllably changing the mechanical impedance of the piezoelectric layer according to the frequency of an electrical message signal arriving from an electronic member, such as a sensor. Third and fourth electrodes may be attached to the piezoelectric layer and the electrical circuit, such that the switching element alternately connects the electrodes in parallel and anti-parallel electrical connections so as to controllably change the mechanical impedance of the piezoelectric layer.

Furthermore, medical implant 2 may be that described in commonly owned U.S. Pat. No. 6,277,078 to Porat, et al, “System and method for monitoring a parameter associated with the performance of a heart,” incorporated herein by reference. U.S. Pat. No. 6,277,078 describes an intrabody implantable system for long-term, real time monitoring of at least one parameter associated with heart performance. The system includes (a) a first sensor being implantable within a heart and being for collecting information pertaining to a pressure in a first cavity of the heart; (b) at least one additional sensor being implantable in a blood vessel supporting blood flow into or out of a second cavity of the heart, the at least one additional sensor being for collecting information pertaining to a pressure and a flow within the blood vessel; and (c) at least one device implantable in the body and being in data communication with the first sensor and the at least one additional sensor, the at least one device being for receiving the information pertaining to the pressure in the first cavity of the heart and the information pertaining to the pressure and the flow within the blood vessel and for relaying the information pertaining to the pressure in the first cavity of the heart and the information pertaining to the pressure and the flow within the blood vessel outside the body.

In accordance with U.S. Pat. No. 6,277,078, the at least one device includes at least one transducer for converting electric signal into a radiative signal, wherein the radiative signal is selected from the group consisting of radio frequency, a magnetic field, an electric field and acoustic radiation. For example, the at least one transducer may be an acoustic transducer and the radiative signal may be an acoustic signal. Alternatively, the at least one transducer may be a magnetic field transducer and the signal may be a magnetic field signal.

Additionally, in accordance with U.S. Pat. No. 6,277,078, the system may include at least one power source, preferably integrated into the at least one device and preferably, arranged as an at least one energizeable power source. The at least one energizeable power source includes at least one transducer for converting a radiative energy into electric energy.

Furthermore, in accordance with U.S. Pat. No. 6,277,078, the radiative energy for energizing the energizeable power source is selected from the group consisting of radio frequency, a magnetic field, an electric field and acoustic radiation. For example, the at least one transducer may be an acoustic transducer and the radiative energy may be an acoustic energy. Alternatively, the transducer may be a magnetic field transducer and the radiative energy may be a magnetic field.

Additionally, medical implant 2 may be that described in commonly owned U.S. Pat. No. 6,237,398 to Porat, et al., “System and method for monitoring pressure, flow and constriction parameters of plumbing and blood vessels,” incorporated herein by reference. U.S. Pat. No. 6,237,398 describes a system and method of quantifying flow, detecting a location of an obstruction and quantifying a degree of the obstruction in a pipe characterized in pulsatile flow. The method includes the steps of (a) attaching at least two spaced pressure sensors onto inner walls of the pipe; (b) using the at least two spaced pressure sensors for recording pressure records associated with each of the at least two pressure sensors within the pipe; and (c) using the pressure records for quantifying the pulsatile flow in the pipe, for detecting the location of the obstruction in the pipe and for quantifying the degree of the obstruction in the pipe.

In accordance with the present invention, medical implant 2 may be capable of telemetric communication with an extracorporeal device.

For example, medical implant 2 may be that described in commonly owned U.S. Pat. No. 6,198,965 to Penner, et al., “Acoustic telemetry system and method for monitoring a rejection reaction of a transplanted organ,” incorporated herein by reference. U.S. Pat. No. 6,198,965 describes a telemetry system for monitoring a rejection reaction of a transplanted organ being transplanted within a patient's body. The telemetry system includes (a) a telemetry control unit located outside the body of the patient; and (b) a telemetry monitoring unit implanted within the body of the patient, the telemetry monitoring unit including: (i) at least one acoustic transducer being for receiving an acoustic signal from the telemetry control unit and converting the acoustic signal into a first electrical signal, the at least one acoustic transducer further being for receiving a second electrical signal and converting the second electrical signal into a transmitted acoustic signal receivable by the telemetry monitoring unit; and (ii) a plurality of electrodes positionable in intimate contact with, or deep within, the transplanted organ and being in communication with the at least one acoustic transducer, the plurality of electrodes being for passing the first electrical signal through the transplanted organ for monitoring the electrical impedance thereof and further being for relaying the second electrical signal corresponding to the electrical impedance to the at least one acoustic transducer so as to enable the monitoring of the presence or absence of the rejection reaction.

Additionally, medical implant 2 may be that described in commonly owned U.S. Pat. No. 6,239,724, to Doron et al., “System and method for telemetrically providing intrabody spatial position,” incorporated herein by reference. U.S. Pat. No. 6,239,724 describes a telemetry system and method for providing spatial positioning information from within a patient's body. The system includes at least one implantable telemetry unit which includes (a) at least one first transducer being for converting a power signal received from outside the body, into electrical power for powering the at least one implantable telemetry unit; (b) at least one second transducer being for receiving a positioning field signal being received from outside the body; and (c) at least one third transducer being for transmitting a locating signal transmittable outside the body in response to the positioning field signal.

In accordance with the present invention, the implant system may be designed in accordance with the teaching of commonly owned U.S. patent application, “Implant System,” to Yarden, O. and Fastovsky V., filed concurrently with the present application, and incorporated herein by reference.

The implant system may thus include a self-expansible structure for implantation in a body cavity. The structure may have a generally tubular outline, a nominal length, a nominal diameter, and a unique behavior under constraint. The unique behavior may be expressed by a transitional diameter, at which a constrained behavior of the structure changes so that, at a constraining diameter smaller than the transitional diameter, the structure conforms to the constraint by decreasing the structure's diameter, to below the nominal diameter, and elongating beyond the nominal length, while at a constraining diameter larger than the transitional diameter but smaller than the nominal diameter, the structure conforms to the constraint by decreasing the structure's diameter below the nominal diameter, while substantially maintaining the nominal length. The structure may be operable for insertion into a body cavity, via a catheter, having a catheter inner diameter smaller than the transitional diameter, by decreasing the structure's diameter, and elongating, and the structure may be operable for implantation in the body cavity, having a cavity inner diameter greater than the transitional diameter but smaller than the nominal diameter, by decreasing the structure's nominal diameter, while substantially maintaining the nominal length.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

It will therefore be appreciated that while the invention has been described with respect to several preferred embodiments, these are set forth merely for purposes of example, and that many other variations, modifications and applications of the invention may be made.

What is claimed is:

1. A deployment device for deploying an implant, said implant being or having a self-expansible anchoring element, at a target location in a body cavity, comprising:
an outer tube;
an inner tube being translatably accepted in said outer tube and being designed for receiving the implant thereon,
such that said self-expansible anchoring element is in a contracted condition when enclosed by said outer tube, expansible to a partially-expanded condition when received on said inner tube and the outer tube is retracted, and further expansible to a fully-expanded condition when the inner tube is removed.

2. The device according to claim 1, wherein said inner tube is designed to translatably accept a guide wire therethrough.

3. The device according to claim 1, wherein said device further includes a retainer, to prevent the implant from sliding along the inner tube, together with the retracting outer tube.

4. The device according to claim 3, wherein said retainer is an intermediate tube located between said inner and outer tubes.

5. The device according to claim 1, wherein receiving the implant on the inner tube is effectable by fixing at least a portion of said anchoring element around said inner tube, so as to restrict the implant to the partially-expanded condition of the anchoring element when the implant is free of other restraining forces.

6. The device according to claim 5, wherein fixing is selected from the group consisting of wrapping, coiling, hooking, inserting into slots, and a combination thereof.

7. The device according to claim 1, further comprising a guide wire to be passed through said inner tube for guiding said implant, inner tube, and outer, tube, to the target location in the body cavity.

8. The device according to claim 1, further comprising a balloon which is inflatable to a size corresponding to the size of the body cavity at said target location to thereby facilitate locating the implant, inner tube, and outer tube, at said target location.

9. The device according to claim 1, wherein said lumen is a blood vessel, and said device further includes a balloon which is inflatable to propel the implant, inner tube, and outer tube, by blood flow to the target location within the blood vessel.

10. The device according to claim 9, wherein said balloon is fixed to the distal end of said outer tube.

11. The device of claim 1, wherein said implant comprises a power source.

12. The device of claim 1, wherein said implant comprises an extracorporeally energizeable power source.

13. The device of claim 1, wherein said implant is capable of telemetric communication with an extracorporeal device.

14. The device of claim 1, wherein said implant being or having a self-expansible anchoring element is a stent.

15. A deployment system for deploying an implant at a target location in a body cavity, comprising:
an outer tube;
an inner tube being translatably accepted in said outer tube; and
an implant received on said inner tube and enclosed by said outer tube;
said implant being or having a self-expansible anchoring element which is in a contracted condition when enclosed by said outer tube, expands to a partially-expanded condition when the outer tube is retracted, and expands to a fully-expanded condition when the inner tube is removed.

16. The system according to claim 15, wherein said inner tube is designed to translatably accept a guide wire therethrough.

17. The system according to claim 15, wherein said device further includes a retainer, to prevent the implant from sliding along the inner tube, together with the retracting outer tube.

18. The system according to claim 17, wherein said retainer is an intermediate tube located between said inner and outer tubes.

19. The system according to claim 15, wherein at least a portion of said anchoring element is fixed to said inner tube, so as to restrict the implant to the partially-expanded condition of the anchoring element when the implant is free of other restraining forces.

20. The system according to claim 19, wherein the manner by which said anchoring element is fixed to said inner tube is selected from the group consisting of wrapping, coiling, hooking, inserting into slots, and a combination thereof.

21. The system according to claim 19, wherein said anchoring element is or includes an elastic spring wire.

22. The system according to claim 19, wherein said anchoring element is or includes an elastic spring leaf.

23. The system according to claim 15, further comprising a guide wire to be passed through said inner tube for guiding said implant, inner tube, and outer tube, to the target location in the body cavity.

24. The system according to claim 15, further comprising a balloon which is inflatable to a size corresponding to the size of the body cavity at said target location to thereby facilitate locating the implant, inner tube, and outer tube, at said target location.

25. The system according to claim 15, wherein said lumen is a blood vessel, and said device further includes a balloon which is inflatable to propel the implant, inner tube, and outer tube, by blood flow to the target location within the blood vessel.

26. The system according to claim 25, wherein said balloon is fixed to the distal end of said outer tube.

27. The system of claim 15, wherein said implant comprises a power source.

28. The system of claim 15, wherein said implant comprises an extracorporeally energizeable power source.

29. The system of claim 15, wherein said implant is capable of telemetric communication with an extracorporeal device.

30. The system of claim 15, wherein said implant being or having a self-expansible anchoring element is a stent.

31. A method of deploying an implant at a target location in a body cavity, comprising: introducing a deployment system according to claim 15 into the body cavity and maneuvering said deployment system to said target location in the body cavity;
retracting said outer tube with respect to said implant such that the anchoring element of the implant self-expands from its contracted condition to its partially-expanded condition; and
withdrawing said inner tube from the implant such that the anchoring element of the implant self-expands from its partially-expanded condition to its fully-expanded condition and becomes deployed at said target location within said body cavity.

32. The method according to claim 31, wherein, after retracting said outer tube to self-expand the anchoring element from its contracted condition to its partially-expanded condition, said inner tube is manipulated to adjust the position or orientation of the implant within the body cavity before withdrawing said inner tube to fully-expand the anchoring element.

33. The method according to claim 31, wherein said deployment device is guided to said target location in the body cavity by a guide wire passed through said inner tube.

34. The method according to claim 3 1, wherein said deployment device is located at said target location by a balloon which is inflated to a size corresponding to the size of the body cavity at said target location.

35. The method according to claim 31, wherein said balloon is located at the distal end of said outer tube.

36. The method according to claim 31, wherein said lumen is a blood vessel, and said method further includes a balloon which is inflated to propel the implant, inner tube, and outer tube, by the blood flow to the target location within the blood vessel.

37. The method according to claim 31, wherein said implant is retained on the inner tube, upon retraction of the outer tube, by a retainer.

38. The method according to claim 31, wherein a portion of said anchoring element is fixed to said inner tube in the partially-expanded condition of the anchoring element when the implant is enclosed by said outer tube.

39. The method according to claim 38, wherein the manner by which said anchoring element is fixed to said inner tube is selected from the group consisting of wrapping, coiling, hooking, inserting into slots, and a combination thereof.

40. The method according to claim 34, wherein said anchoring element is or includes an elastic spring wire.

41. The method according to claim 34, wherein said anchoring element is or includes an elastic spring leaf.

42. The method of claim 31, wherein said implant comprises a power source.

43. The method of claim 31, wherein said implant comprises an extracorporeally energizeable power source.

44. The method of claim 31, wherein said implant is capable of telemetric communication with an extracorporeal device.

45. The method of claim 31, wherein said implant being or having a self-expansible anchoring element is a stent.
